(19) United States
(12) Patent Application Publication
(10) Pub. No.: US 2026/0234203 A1
(43) Pub. Date: Aug. 13, 2026

(54) COMPOSITIONS AND METHODS FOR INHIBITION OF DEGRANULATION OF NEUTROPHIL CELLS

(71) Applicants: UNIVERSITY OF LOUISVILLE RESEARCH FOUNDATION, INC., Louisville, KY (US); DEGRANIN THERAPEUTICS, LLC, Louisville, KY (US)

(72) Inventors: Kenneth R. MCLEISH, Louisville, KY (US); Jiapeng HUANG, Louisville, KY (US); Madhavi J. RANE, Louisville, KY (US); Silvia M. URIARTE, Louisville, KY (US); Jun YAN, Louisville, KY (US)

(73) Assignees: UNIVERSITY OF LOUISVILLE RESEARCH FOUNDATION, INC., Louisville, KY (US); DEGRANIN THERAPEUTICS, LLC, Louisville, KY (US)

(21) Appl. No.: 19/416,006

(22) Filed: Dec. 11, 2025

Related U.S. Application Data
(60) Provisional application No. 63/730,700, filed on Dec. 11, 2024.

(51) Int. Cl. C07K 14/47; A61K 38/00; A61P 37/02; C07K 7/06

(52) U.S. Cl. CPC C07K 14/4702; A61P 37/02; C07K 7/06; A61K 38/00; C07K 2319/00

(57) ABSTRACT

In certain aspects, provided herein are fusion proteins comprising a TAT-fusion protein comprising an 11 amino acid cell penetrating peptide TAT operably linked to an amino acid N-terminal SNARE domain of SNAP-23, wherein the N-terminal SNARE domain of SNAP-23 is about 30-70 amino acids in length. Also, in certain aspects, provided herein are neutrophil degranulation comprising administering a fusion protein comprising a TAT-fusion protein comprising an 11 amino acid cell penetrating peptide TAT operably linked to a 33 amino acid N-terminal SNARE domain of SNAP-23 or a pharmaceutical composition comprising this fusion protein, to a patient in need thereof, wherein the inhibition is compared to a control.

### CROSS-REFERENCE TO RELATED APPLICATION

This application claims priority to U.S. Provisional Application No. 63/730,700 that was filed on Dec. 11, 2024. The entire content of the application referenced above is hereby incorporated by reference herein.

### STATEMENT OF GOVERNMENT RIGHTS

This invention was made with government support under R43 HL169129 awarded by the National Institutes of Health. The government has certain rights in the invention.

This invention was made with an award from the Kentucky Cabinet for Economic Development, under Grant Agreement CED No. 041-2023-001-003.

### SEQUENCE LISTING

The instant application contains a Sequence Listing which has been submitted electronically in XML format and is hereby incorporated by reference in its entirety. Said XML copy, created on Mar. 27, 2026, is named 17541092US1, and is 21,793 bytes in size.

### BACKGROUND

Acute respiratory distress syndrome (ARDS) is a severe form of acute lung injury (ALI) caused by uncontrolled inflammatory damage to the alveolar-capillary barrier, resulting in increased vascular permeability, pulmonary edema, and hypoxemia. ARDS occurs in 10% of intensive care unit (ICU) admissions and accounts for 25% of patients requiring mechanical ventilation. Prior to the COVID-19 pandemic, between 200,000 and 250,000 cases/year of ARDS were diagnosed in the US. Those cases were caused by bacterial and viral pneumonia, non-pulmonary sepsis, aspiration, trauma, and pancreatitis. ARDS remains underrecognized in clinical practice, with up to 40% of cases reportedly missed. During the height of the COVID-19 pandemic, the number of ARDS patients in the US increased to an estimated 500,000 cases/year, and over 3 million patients worldwide developed ARDS. Although the prevalence of COVID-19 ARDS has declined, the total number of ARDS patients is projected to grow at a compound annual growth rate (CAGR) of 6-10% over the next decade. The cost of caring for each ARDS patient ranges from $70,000 to $400,000, resulting in an estimated US market size of $1.6 billion in 2023, and the CAGR is forecasted to be 5% to 7%, over the next decade. While incidence of ARDS diagnosis is less in Europe and Japan, cost is similar.

Introduction of enhanced supportive measures initially improved outcomes, however, ARDS-related mortality has remained stable at 35%-40% for the past several years. To date, no FDA-approved pharmacologic intervention for ARDS exists. Barriers to development of effective therapy, include heterogeneity of etiology, pathophysiology, and severity of acute lung injury (ALI).

Accordingly, effective immunomodulatory treatments for ARDS are urgently needed.

### SUMMARY

In certain aspects, provided herein is a fusion protein comprising a TAT-fusion protein comprising an 11 amino acid cell penetrating peptide TAT operably linked to an amino acid N-terminal SNARE domain of SNAP-23, wherein the N-terminal SNARE domain of SNAP-23 is about 30 to 70 amino acids in length. In certain embodiments, the N-terminal SNARE domain of SNAP-23 is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids in length.

In certain aspects, the cell penetrating peptide TAT is amino acid sequence

(SEQ ID NO: 5)
YGRKKRRQRRR.

In certain aspects, the N-terminal SNARE domain of SNAP-23 is amino acid sequence

(SEQ ID NO: 16)
AHQITDESLESTRRILGLAIESQDAGIKTITML,
(SEQ ID NO: 17)
MAHQITDESLESTRRILGLAIESQDAGIKTITML
or
(SEQ ID NO: 18)
MAHQITDESLESTRRILGLAIESQDAGIKTITMLDEQKEQLNRIEEGLDQ
INKDMRETEKTLTELNK.

In certain aspects, the protein is amino acid sequence

(SEQ ID NO: 15)
YGRKKRRQRRRAHQITDESLESTRRILGLAIESQDAGIKTITML.

In certain aspects, provided herein is a pharmaceutical composition comprising the fusion protein described herein and a pharmaceutically acceptable excipient.

In certain aspects, provided herein is a method inhibiting release of intracellular granule contents (degranulation) by neutrophils comprising administering the fusion protein described herein or the pharmaceutical composition comprising the fusion protein described herein, to a patient in need thereof, wherein the inhibition is compared to a control.

In certain aspects, provided herein is a method inhibiting generation of reactive oxygen species (ROS) by neutrophils comprising administering the fusion protein described herein or the pharmaceutical composition comprising the fusion protein described herein, to a patient in need thereof, wherein the inhibition is compared to a control.

In certain aspects, the patient has acute respiratory distress syndrome (ARDS).

In certain aspects, the patient is infected with severe acute respiratory syndrome coronavirus 2 (SARS-COV-2).

In certain aspects, provided herein is a fusion protein described herein or a pharmaceutical composition comprising the fusion protein described herein for use in medical therapy.

### BRIEF DESCRIPTION OF DRAWINGS

FIG. 1. TAT fusion proteins inhibit degranulation stimulated by COVID-19 plasma. Results show the increase in plasma membrane markers of secretory vesicle (CD35) and gelatinse granules (CD11b). Mean fluorescent intensity of each marker was determined by flow cytometry. * p<0.05, ** p<0.01 ***, p<0.001, **** p<0.0001. n.s. not significant.

FIG. 2. TAT-fusion proteins inhibit priming of respiratory burst activity by COVID-19 plasma and BALF. Superoxide release stimulated by fMLF in TNF-primed and unprimed neutrophils from healthy subject was compared to that stimulated with 10% plasma or BALF obtained from critical COVID-19 patients. Pre-treatment with a combination of TAT-SNAP-23 (also called “DGN-23”) and TAT-STX-4 inhibited superoxide release of naïve neutrophils incubated with COVID-19 plasma or BALF and then stimulated with fMLF. * p<0.05, **** p<0.0001.

FIGS. 3A-3B. COVID-19 Plasma Induces Net Formation. FIG. 3A shows a representative confocal image of NETs, detected by DAPI and lactoferrin staining, after a 2 hr incubation of naïve neutrophils with 10% COVID-19 plasma. FIG. 3B shows quantitative analysis of confocal images by IMARIS software; from 3 independent donors.

FIGS. 4A-4C. TAT-Fusion Proteins Inhibit Bacterium-induced NET Formation. FIG. 4A shows a representative confocal image of NETs induced by CFSE-labelled P. stomatis in neutrophils pre-treated with or without a combination of DGN-23 (1 mg/ml) and TAT-STX-4 (1 μg/ml). FIG. 4B shows the quantification of NETs by colocalization of DAPI and lactoferrin staining by IMARIS software, n=3. FIG. 4C shows inhibition of secretory vesicles (CD35) and specific granule (CD66b) degranulation in neutrophils from the same donors used in NET experiments. DGN-23 and TAT-STX-4, but not TAT-Control, inhibited degranulation stimulated by P. stomatis and fMLF.

FIG. 5. Neutrophils are stimulated to undergo degranulation in vivo in COVID-19 patients.

FIG. 6. Modified DGN-23, containing amino acids 13-45 of the SNARE domain (termed DGN-1345), inhibited neutrophil degranulation with concentration inhibition curves similar to the parent compound.

FIG. 7. Results of incubation of cultured HK-11 cells with DGN-1345 for 10 to 60 min at concentrations of 0 nM to 900 nM.

FIGS. 8A-8G Administration of DGN-23 to mice infected with SARS-COV-2 significantly inhibited lung injury; determined by histology, BALF protein content, lung edema determined by lung weight, and lung epithelial cell damage determined by BALF RAGE content; and significantly inhibited neutrophil degranulation in lungs, determined by BALF MMP9 and elastase content.

### DETAILED DESCRIPTION

Barriers to development of effective therapy include heterogeneity of etiology, pathophysiology, and severity of acute lung injury (ALI).

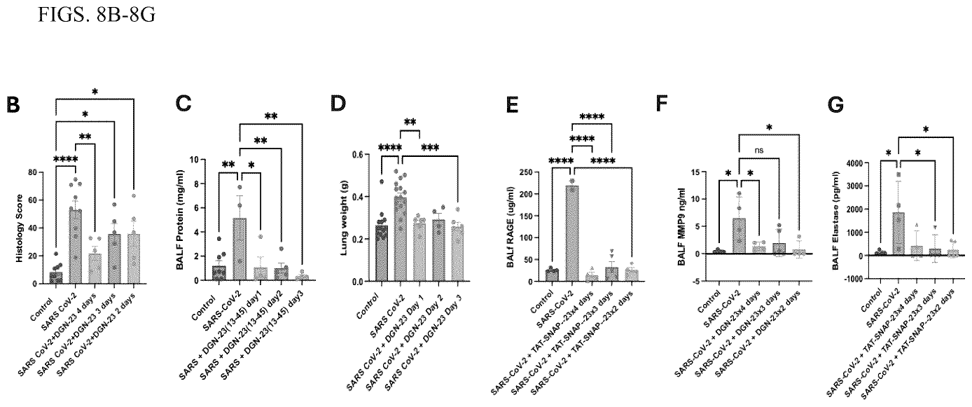

Despite those barriers, one pathophysiologic mechanism common to ARDS is an uncontrolled inflammatory response resulting from neutrophil-mediated vascular endothelial cell or alveolar epithelial cell injury. Scientifically rigorous evidence supports a causative role for extracellular release of neutrophil anti-microbial agents in ARDS, including that caused by COVID-19. Degranulation is one critical mechanism by which toxic anti-microbials are released. Support for a causative role of degranulation includes: 1) neutrophils isolated from patients with ARDS show enhanced degranulation, 2) high concentrations of neutrophil granule constituents are found in blood and bronchoalveolar lavage fluid (BALF), and 3) injection of neutrophil granule constituents into lungs of neutrophil-depleted animals causes ALI. Those studies support targeting neutrophil degranulation as a promising treatment strategy. Our pre-clinical studies with the original inhibitor, TAT-SNAP-23 (also referred to as “DGN-23”), showed that inhibition of degranulation reduced lung injury in rodent models of ARDS, including a model of SARS-COV-2 infection shown in FIGS. 8A-8G, confirming that treatment strategy.

ARDS can be caused by severe acute respiratory syndrome coronavirus 2 (SARS-COV-2), which is a novel viral pathogen that causes a clinical disease called coronavirus disease 2019 (COVID-19). Approximately 20% of infected patients experience a severe manifestation of the disease, causing bilateral pneumonia and acute respiratory distress syndrome (ARDS). Severe COVID-19 patients also have a pronounced coagulopathy with approximately 30% of patients experiencing thromboembolic complications.

Current therapy for severe and critical COVID-19 is inadequate. Although mortality rates are declining as more is learned about treatment of critically ill COVID-19 patients, in early January 2021, over 375,000 people had died in the U.S. alone and deaths continue to occur. Evidence suggests that neutrophils play a significant role in the lung injury and blood vessel clotting that are primarily responsible for deaths and disabilities.

Neutrophils are potential drivers of acute lung injury and increased blood clot formation (hypercoagulability) caused by severe infections with SARS-COV-2 leading to COVID-19. Neutrophils contribute to COVID-19 by generating reactive oxygen species (ROS), release of intracellular granule contents (degranulation), and formation of neutrophil extracellular traps (NETs). Sera from COVID-19 patients showed induction of NETs. Degranulation not only releases toxic chemicals but also contributes to priming for enhanced ROS generation by cytokines that are also produced during the COVID-19 cytokine storm. The data presented below show that degranulation also plays a role in NET formation. Thus, degranulation is a critical event in all neutrophil responses leading to COVID-19, as well as other forms of ARDS. The findings presented herein indicate that inhibition of neutrophil degranulation is a potential therapeutic approach for severe or critical COVID-19, in that ROS production, release of proteolytic enzymes, and NET formation are all inhibited.

Due to unreliable production in terms of yield and potency of TAT-SNAP-23 (also called “DGN-23”) by bacteria, however, modifications were made to DGN-23. These modifications resulted in changes in amino acid composition to remove amino acids not contributing to the function of the protein, and changes to the active portion of the molecule by removing amino acids 1-12 and 46-78. The new recombinant protein is called “DGN-1345.” This new protein product is 44 amino acids in length, possesses potency of inhibition of degranulation equivalent to the parent compound, and can be reliably produced at a higher yield. The amino acid composition of DGN-23 was modified to eliminate portions that would interfere with drug development and the SNARE domain of SNAP-23 (which is the active portion of the molecule) was altered to enhance production reliability and improve potency.

DGN-1345 inhibits neutrophil degranulation, a process that contributes to tissue injury in acute lung injury leading to acute respiratory distress syndrome (ARDS) and in other inflammatory diseases.

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the following terms are believed to be well understood by one of ordinary skill in the art, definitions are set forth to facilitate explanation of the presently disclosed subject matter.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Some of the polynucleotide and polypeptide sequences disclosed herein are cross-referenced to GENBANK® accession numbers. The sequences cross-referenced in the GENBANK® database are expressly incorporated by reference as are equivalent and related sequences present in GENBANK® or other public databases. Also expressly incorporated herein by reference are all annotations present in the GENBANK® database associated with the sequences disclosed herein. Unless otherwise indicated or apparent, the references to the GENBANK® database are references to the most recent version of the database as of the filing date of this Application.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

The term “isolated”, when applied to a nucleic acid or polypeptide, denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high performance liquid chromatography (HPLC). A polypeptide that is the predominant species present in a preparation is substantially isolated. The term “isolated” further denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 99% pure.

The terms “polypeptide”, “protein”, and “peptide”, which are used interchangeably herein, refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment,” when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified variants thereof (e.g., degenerate variants, including degenerate codon substitutions) and complementary sequences and as well as the sequence explicitly indicated.

The term “degenerate variant” refers to a nucleic acid having a residue sequence that differs from a reference nucleic acid by one or more degenerate codon substitutions. Degenerate codon substitutions can be achieved by generating sequences in which the third position of one or more selected (or all) codons is substituted with mixed base and/or deoxyinosine residues.

Degranulation (also called “exocytosis”) in neutrophils occurs in sequential stages, starting with disassembly of the cortical actin cytoskeleton and granule recruitment to the plasma membrane, where tethering and docking of granules are mediated by specific proteins. This tethering and docking of the granules to specific proteins is then followed by membrane fusion and release of an assortment of granules, including specific, secretory, azurophil, and gelatinase granules, as well as their related contents into the extracellular media.

Soluble N-ethylmaleimide-sensitive factor attachment protein receptors (“SNAREs” or “SNAP receptors”) perform a central role in neutrophil degranulation by mediating granule docking and membrane fusion. SNAREs are classified based on whether a conserved glutamine (Q) or arginine (R) residue is present in their SNARE-interaction motif, an amino acid domain in the SNAREs that mediates the association of the various SNARE proteins into a core complex capable of mediating granule docking and membrane fusion. The SNARE hypothesis proposes that a syntaxin protein provides one Q-containing helix, a soluble N-ethylmaleimide-sensitive factor attachment protein-23/25 (SNAP-23/25) contributes two Q-containing helices, and a vesicle-associated membrane protein (VAMP) contributes one R-containing helix to a coiled-coil trans-SNARE complex. This 3Q: 1R helix allows specific SNARE pairing and provides the energy for membrane fusion between the vesicle and the target membrane.

SNAP-23 is a SNAP-25 homolog that is expressed in non-neuronal tissue. At the mRNA level, five SNAP-23 isoforms have been reported in eosinophils, basophils, neutrophils and peripheral blood mononuclear cells. In human neutrophils, SNAP-23 a is the major form of this SNARE protein that is expressed. SNAP-23 has been specifically detected on gelatinase granules, specific granules, and the plasma membrane. Similarly, it has been shown that introduction of anti-syntaxin 4 antibodies into neutrophils by electroporation was capable of inhibiting specific and gelatinase granule degranulation stimulated by Ca²⁺ and GTPγS. As such, SNAP-23, syntaxin 4, and syntaxin 6 are potential targets for inhibition of degranulation of neutrophil granule subsets.

In some embodiments of the presently disclosed subject matter, compositions are provided that comprise isolated polypeptides for inhibiting degranulation of neutrophil granules, and thereby decreasing detrimental neutrophil-mediated inflammatory responses. In some embodiments, an isolated fusion polypeptide is provided that comprises a cell-penetrating polypeptide, which facilitates entry of the fusion polypeptide into a neutrophil, and a SNARE polypeptide aptamer, which blocks the interaction of cognate SNARE partners and thereby inhibits SNARE-associated degranulation in neutrophils.

One aspect of the presently disclosed subject matter thus pertains to fusion proteins and nucleic acids (e.g., DNA) encoding the fusion proteins. The term “fusion protein” is intended to describe at least two polypeptides, typically from different sources, which are operatively linked. With regard to the polypeptides, the term “operatively linked” is intended to mean that the two polypeptides are connected in a manner such that each polypeptide can serve its intended function. Typically, the two polypeptides are covalently attached through peptide bonds and can be produced by standard recombinant or chemical synthesis techniques. For example, using recombinant techniques, a DNA molecule encoding a first polypeptide can be ligated to another DNA molecule encoding the second polypeptide, and the resultant hybrid DNA molecule can be expressed in a host cell to produce the fusion protein. The DNA molecules are generally ligated to each other in a 5′ to 3′ orientation such that, after ligation, the translational frame of the encoded polypeptides is not altered (i.e., the DNA molecules are ligated to each other in-frame).

In some embodiments, the fusion polypeptides of the presently disclosed subject matter are comprised, in part, of a first polypeptide, referred to as a cell-penetrating polypeptide. The term “cell-penetrating polypeptide” is used herein to refer to polypeptides that have the ability to provide entry of a coupled peptide into a cell. Exemplary cell-penetrating polypeptides that can be used in accordance with the presently-disclosed subject matter include, but are not limited to: a human immunodeficiency virus transactivator of transcription (TAT) polypeptide; an Antennapedia homeodomain polypeptide, referred to as “penetratin” (e.g., AKIWFQNRRMKWKKEN; SEQ ID NO: 6); an HSV VP22 polypeptide (SEQ ID NO: 7); a polyarginine polypeptide (e.g., RRRRRRRRR; SEQ ID NO: 8); a pep-1 polypeptide (KETWWETWWTEWSQPKKKRKV; SEQ ID NO: 9); and a transportan polypeptide (GWTLNSAGYLLGKINLKALAALAKKIL; SEQ ID NO: 10). In some embodiments, the cell-penetrating peptide thus comprises a TAT, penetratin, HSV VP22, a polyarginine, a pep-1, or a transportan polypeptide. In some embodiments, the cell-penetrating polypeptide is a TAT polypeptide and has the following amino acid sequence: YGRKKRRQRRR (SEQ ID NO: 5). In some embodiments, the cell-penetrating polypeptide can be flanked by glycine residues to allow for free rotation.

In some embodiments, the first polypeptide of the fusion protein is operatively linked to a second polypeptide, which is a SNARE polypeptide aptamer. The term “aptamer” is used herein to refer to a fragment of an endogenous protein that is capable of binding to cognate protein binding sites and preventing interaction with target molecules. For example, in some embodiments, the SNARE polypeptide aptamer selectively binds a SNARE-interaction motif of a target SNARE protein such that the SNARE polypeptide aptamer inhibits binding of other SNARE proteins to the target SNARE protein, and thereby inhibits the formation of a trans-SNARE complex.

In some embodiments, the SNARE polypeptide aptamer comprises a SNAP-23 amino-terminus polypeptide fragment or a syntaxin 4 (STX-4) polypeptide fragment, such as the one provided in SEQ ID NO: 14. In other embodiments, the SNARE polypeptide aptamer is a polypeptide fragment from the amino-terminus of SNAP-23 (e.g., human SNAP-23; GENBANK® Accession No. NP_003816), such as the polypeptide provided in SEQ ID NO: 1. In some embodiments, the SNARE polypeptide aptamer comprises a polypeptide of SEQ ID NO: 16 and the cell-penetrating polypeptide comprises a polypeptide of SEQ ID NO: 5.

Particular TAT-fusion protein inhibitors of neutrophil degranulation contain the 11 amino acid cell penetrating peptide, TAT, and the N-terminal SNARE domain of SNAP-23 (TAT-SNAP-23, also called “DGN-23”) or the SNARE domain of syntaxin-4 (TAT-STX-4). See, for example, U.S. Pat. No. 8,709,758, which is incorporated by reference herein.

### Exemplary SNARE Polypeptide Aptamer Sequences

SEQ ID NO: 1 is an amino acid sequence of a SNAP-23 polypeptide that includes 78 amino acids from the N-terminus of the full-length human SNAP-23 protein.

```
Met Asp Asn Leu Ser Ser Glu Glu Ile Gln Gln Arg Ala His Gln Ile
1 5 10 15
Thr Asp Glu Ser Leu Glu Ser Thr Arg Arg Ile Leu Gly Leu Ala Ile
                        20 25 30
Glu Ser Gln Asp Ala Gly Ile Lys Thr Ile Thr Met Leu Asp Glu Gln
                35 40 45
Lys Glu Gln Leu Asn Arg Ile Glu Glu Gly Leu Asp Gln Ile Asn Lys
        50 55 60
Asp Met Arg Glu Thr Glu Lys Thr Leu Thr Glu Leu Asn Lys
65 70 75
```

SEQ ID NO:2 is an amino acid sequence of a SNAP-23 polypeptide that includes 40 amino acids from the N-terminus of the full-length human SNAP-23 protein.

```
Thr Ile Thr Met Leu Asp Glu Gln Lys Glu Gln Leu Asn Arg Ile Glu
1 5 10 15
Glu Gly Leu Asp Gln Ile Asn Lys Asp Met Arg Glu Thr Glu Lys Thr
                        20 25 30
Leu Thr Glu Leu Asn Lys Cys Cys
                35 40
```

SEQ ID NO:3 is an amino acid sequence of a SNAP-23 polypeptide that includes 60 amino acids from the C-terminus of the full-length human SNAP-23 protein.

```
Met Glu Glu Asn Leu Thr Gln Val Gly Ser Ile Leu Gly Asn Leu Lys
1 5 10 15
Asp Met Ala Leu Asn Ile Gly Asn Glu Ile Asp Ala Gln Asn Pro Gln
                        20 25 30
Ile Lys Arg Ile Thr Asp Lys Ala Asp Thr Asn Arg Asp Arg Ile Asp
                35 40 45
Ile Ala Asn Ala Arg Ala Lys Lys Leu Ile Asp Ser
        50 55 60
```

SEQ ID NO:4 is an amino acid sequence of a SNAP-23 polypeptide that includes 24 amino acids from the C-terminus of the full-length human SNAP-23 protein.

Thr Asp Lys Ala Asp Thr Asn Arg Asp Arg Ile Asp Ile Ala Asn Ala
1 5 10 15
Arg Ala Lys Lys Leu Ile Asp Ser
20

SEQ ID NO:5 is an amino acid sequence of a human immunodeficiency virus transactivator of transcription (TAT) cell-penetrating polypeptide.

Tyr Gly Arg Lys Lys Arg Arg Gln Arg Arg Arg
1 5 10

SEQ ID NO: 14 is an amino acid sequence of a syntaxin 4 polypeptide aptamer.

```
Val Thr Arg Gln Ala Leu Asn Glu Ile Ser Ala Arg His Ser Glu Ile
1 5 10 15
Gln Gln Leu Glu Arg Ser Ile Arg Glu Leu His Asp Ile Phe Thr Phe
                        20 25 30
Leu Ala Thr Glu Val Glu Met Gln Gly Glu Met Ile Asn Arg Ile Glu
                35 40 45
Lys Asn Ile Leu Ser Ser Ala Asp Tyr Val Glu Arg Gly Gln Glu His
        50 55 60
Val Lys Thr Ala
65
```

SNARE 13-45 is an amino acid sequence of a SNAP-23 polypeptide that includes 33 amino acids from the N-terminus of the full-length human SNAP-23 protein (amino acids 13-45). In this sequence, the first 12 amino acids and the last 33 amino acids from the SNARE domain were removed. It was effective at inhibiting neutrophil degranulation.

(SEQ ID NO: 21)
Arg Ala His Gln Ile Thr Asp Glu Ser Leu Glu Ser
Thr Arg Arg Ile Leu Gly Leu Ala Ile Glu Ser Gln
Asp Ala Gly Ile Lys Thr Ile Thr Met Leu

The following SNARE domain sequences from the 1-78 amino acid sequence derived from the native protein have been tested:

(SEQ ID NO: 22)
SNARE 13-78:
AHQITDESLESTRRILGLAIESQDAGIKTITMLDEQKEQLNRIEEGLDQI
NKDMRETEKTLTELNK

This is the entire SNARE domain minus the first 12 amino acids. It was also effective at inhibiting neutrophil degranulation.

(SEQ ID NO: 23)
SNARE 19-78:
ESLESTRRILGLAIESQDAGIKTITMLDEQKEQLNRIEEGLDQINKDMRE
TEKTLTELNK

This sequence removed the first 18 amino acids from the N-terminus of the SNARE domain. It did not inhibit degranulation.

(SEQ ID NO: 19)
SNARE 46-78:
DEQKEQLNRIEEGLDQINKDMRETEKTLTELNK

This sequence removed the first 45 amino acids from the N-terminus of the SNARE domain. It did not inhibit degranulation.

The terms “N-terminus” or “amino-terminus” and “C-terminus” or “carboxyl-terminus” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide. Where amino-terminus or carboxyl-terminus refer to an entire polypeptide or polypeptide fragment, the terms refer to one or more amino acids at amino or carboxyl ends, respectively, of the polypeptide or the polypeptide fragment.

In some embodiments of the presently disclosed SNARE polypeptide aptamers, one or more amino acid residues can be deleted from the amino-terminus, the carboxyl-terminus, or from both ends of the polypeptide fragments. For example, in some embodiments, about 1, about 2, about 3, about 4, about 5, about 6, about 7, about 8, about 9, or about 10 amino acids can be deleted from the amino-terminus or the carboxyl-terminus of the polypeptide fragments provided in SEQ ID NO: 16. In some embodiments, about 1, about 2, about 3, about 4, about 5, about 6, about 7, about 8, about 9, or about 10 amino acids can be independently deleted from the amino-terminus and the carboxyl-terminus of the polypeptide fragments provided in SEQ ID NO: 16.

As noted, to operatively link the first and second polypeptides, nucleotide sequences encoding the first and second polypeptides are ligated to each other in-frame to create a chimeric gene encoding a fusion polypeptide. In some embodiments, a further nucleic acid sequence encoding an additional polypeptide sequence can be incorporated between the nucleotide sequences encoding the first and second polypeptides. For example, in some embodiments, a fusion polypeptide can be provided that contains an operatively-linked polypeptide, such as an affinity tag, i.e., (cell-penetrating polypeptide)-(affinity tag)-(SNARE polypeptide aptamer).

The term “affinity tag” is used herein to denote a polypeptide segment that can be attached to a one or more polypeptides to provide for purification or detection of the one or more polypeptides. In principal, any peptide or protein for which an antibody or other specific binding agent is available can be used as an affinity tag. Affinity tags include, but are not limited to, a poly-histidine tract, protein A, glutathione S transferase, Glu-Glu affinity tag, substance P, streptavidin binding peptide, or other antigenic epitope, such as a hemagglutinin (HA) polypeptide.

In some embodiments, a fusion polypeptide is provided that comprises an HA polypeptide, such as the polypeptide set forth in SEQ ID NO: 13, as an affinity tag. In some embodiments, a fusion polypeptide is provided that is comprised of an HA polypeptide positioned between a TAT cell-penetrating polypeptide and a SNAP-23 aptamer, such as the polypeptide set forth in SEQ ID NO: 12.

```
SEQ ID NO:12
Met Arg Gly Ser His His His His His His Gly Met Ala Ser Met Thr
1 5 10 15
Gly Gly Gln Gln Met Gly Arg Asp Leu Tyr Asp Asp Asp Asp Lys Asp
                        20 25 30
Arg Trp Gly Ser Lys Leu Gly Tyr Gly Arg Lys Lys Arg Arg Gln Arg
                35 40 45
Arg Arg Gly Gly Ser Thr Met Ser Gly Tyr Pro Tyr Asp Val Pro Asp
        50 55 60
Tyr Ala Gly Ser Met Asp Asn Leu Ser Ser Glu Glu Ile Gln Gln Arg
65 70 75 80
Ala His Gln Ile Thr Asp Glu Ser Leu Glu Ser Thr Arg Arg Ile Leu
                                85 90 95
Gly Leu Ala Ile Glu Ser Gln Asp Ala Gly Ile Lys Thr Ile Thr Met
                        100 105 110
Leu Asp Glu Gln Lys Glu Gln Leu Asn Arg Ile Glu Glu Gly Leu Asp
                115 120 125
Gln Ile Asn Lys Asp Met Arg Glu Thr Glu Lys Thr Leu Thr Glu Leu
        130 135 140
Asn Lys
145
SEQ ID NO: 13
Tyr Pro Tyr Asp Val Pro Asp Tyr Ala
1 5
```

In some embodiments of the presently disclosed subject matter, isolated nucleic acids are further provided that comprise a nucleotide sequence encoding a fusion polypeptide that inhibits neutrophil degranulation. In some embodiments, an isolated nucleic acid is provided that encodes a fusion polypeptide comprising a cell-penetrating polypeptide and a SNARE polypeptide aptamer, which is selected from a SNAP-23 amino-terminus polypeptide fragment, such as the one provided in SEQ ID NO: 1, or a syntaxin 4 polypeptide fragment, such as the one provided in SEQ ID NO: 14. In some embodiments, a nucleic acid sequence is provided that comprises the sequence of SEQ ID NO: 11. In some embodiments, a nucleic acid sequence is provided that encodes a SNARE polypeptide aptamer of SEQ ID NO: 1 and a cell-penetrating peptide of SEQ ID NO: 5.

| SEQ ID NO: 11 | |
|---|---|
| atgcggggtt ctcatcatca tcatcatcat ggtatggcta gcatgactgg tggacagcaa | 60 |
| atgggtcggg atctgtacga cgatgacgat aaggatcgat ggggatccaa gcttggctac | 120 |
| ggccgcaaga aacgccgcca gcgccgccgc ggtggatcca ccatgtcegg ctatccatat | 180 |
| gacgtcccag actatgctgg ctccatggat aatctgtcat cagaagaaat tcaacagaga | 240 |
| gctcaccaga ttactgatga gtctctggaa agtacgagga gaatcctggg tttagccatt | 300 |
| gagtctcagg atgcaggaat caagaccato actatgctgg atgaacaaaa ggaacaacta | 360 |
| aaccgcatag aagaaggctt ggaccaaata aataaggaca tgagagagac agagaagact | 420 |
| ttaacagaac tcaacaaa | 438 |

The nucleic acid sequence for DGN-1345 is as follows:
(SEQ ID NO: 20)
TACGGCCGCAAGAAACGCCGCCAGCGCCGCCGCGCTCACCAGATTACTGATGAGTCTCTGGAAA
GTACGAGGAGAATCCTGGGTTTAGCCATTGAGTCTCAGGATGCAGGAATCAAGACCATCACTAT
GCTG

To generate an exemplary fusion polypeptide in accordance with the presently disclosed subject matter, in some embodiments, the nucleic acid encoding the fusion polypeptide is inserted into an appropriate expression vector that contains the necessary elements for the transcription and translation of the inserted protein-coding sequence. A variety of host-vector systems can be utilized to express an inserted protein-coding sequence, including mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements can be used. As one exemplary embodiment of a vector comprising a nucleic acid sequence of the presently disclosed subject matter, an exemplary vector can be a plasmid, such as the plasmid pTAT-HA, into which a nucleic acid encoding a SNARE polypeptide aptamer can be cloned by the use of internal restriction sites present within the vector.

In some embodiments, the nucleic acids of the presently disclosed subject matter are operably linked to an expression cassette. The terms “associated with”, “operably linked”, and “operatively linked,” when used herein in reference to a nucleic acid sequence, refer to two nucleic acid sequences that are related physically or functionally. For example, a promoter or regulatory DNA sequence is said to be “associated with” a DNA sequence that encodes an RNA or a polypeptide if the two sequences are operatively linked, or situated such that the regulator DNA sequence will affect the expression level of the coding or structural DNA sequence.

The term “expression cassette” refers to a nucleic acid molecule capable of directing expression of a particular nucleotide sequence in an appropriate host cell, and comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression.

Once generated by an appropriate host-vector system, the fusion polypeptides can then be separated and purified by an appropriate combination of known techniques. These methods include, for example: methods utilizing solubility such as salt precipitation and solvent precipitation; methods utilizing the difference in molecular weight, such as dialysis, ultra-filtration, gel-filtration, and SDS-polyacrylamide gel electrophoresis; methods utilizing a difference in electrical charge, such as ion-exchange column chromatography; methods utilizing specific affinity, such as affinity chromatography; methods utilizing a difference in hydrophobicity, such as reverse-phase high performance liquid chromatography; methods utilizing a difference in isoelectric point, such as isoelectric focusing electrophoresis; and, metal affinity columns, such as Ni-NTA. If an operatively linked purification tag, such as HA, is included in the fusion polypeptide, the purification tag can be utilized to purify the fusion polypeptide.

As noted herein, the fusion polypeptides of the presently disclosed subject matter can also be prepared through chemical synthesis according to methods known in the art. Exemplary chemical synthesis methods of producing polypeptides include, but are not limited to, exclusive solid phase synthesis, partial solid phase methods, fragment condensation or classical solution synthesis.

In some embodiments, a cell is provided that comprises a nucleotide sequence that encodes a fusion polypeptide comprised of a cell-penetrating polypeptide and a SNARE polypeptide aptamer in accordance with the presently disclosed subject matter. Nucleic acids containing a target nucleotide sequence (e.g., a nucleotide sequence encoding a fusion polypeptide of the presently disclosed subject matter) operably linked to a regulatory sequence can be introduced into a host cell transiently or, for long term regulation of gene expression, the nucleic acid can be stably integrated into the genome of the host cell or remain as a stable episome in the host cell.

As used herein, the term “host cell” is intended to include any cell or cell line, including prokaryotic and eukaryotic cells, into which a nucleic acid sequence of the presently disclosed subject matter can be introduced and expressed. Exemplary host cells include, but are not limited to, yeast, fly, worm, plant, frog, and mammalian cells. Non-limiting examples of mammalian cell lines which can be used include CHO-cells, 293 cells, or myeloma cells like SP2 or NSO. Other exemplary eukaryotic host cells include insect (e.g., Sp. frugiperda), yeast (e.g., S. cerevisiae, S. pombe, P. pastoris, K. lactis, H. polymorpha), fungal and plant cells. Specific exemplary prokaryotic host cells include E. coli and Bacillus sp.

Nucleic acids comprising a nucleotide sequence of the presently disclosed subject matter operably linked to a regulatory sequence can be introduced into a host cell by standard techniques for transfecting cells. As used herein, the term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells are well-known in the art.

Nucleotide sequences of the presently disclosed subject matter operably linked to a regulatory sequence can be introduced into cells growing in culture by conventional transfection techniques (e.g., calcium phosphate precipitation, DEAE-dextran transfection, electroporation, etc.). In some embodiments, nucleotide sequences can also be transferred into cells in vivo, for example, by application of a delivery mechanism suitable for introduction of nucleic acid into cells in vivo into host production animals, such as retroviral vectors, adenoviral vectors, receptor-mediated DNA uptake, direct injection of DNA or particle bombardment.

Further provided, in some embodiments of the presently disclosed subject matter, are methods for using the fusion polypeptides of the presently disclosed subject matter (i.e., fusion polypeptides comprised of a cell-penetrating polypeptide and a SNARE polypeptide aptamer that is either a SNAP-23 amino terminus polypeptide fragment, such as the one provided in SEQ ID NO: 1, or a syntaxin 4 polypeptide fragment, such as the one provided in SEQ ID NO: 14). In some embodiments, methods for inhibiting neutrophil degranulation are provided. In some embodiments, a method for inhibiting neutrophil degranulation is provided that comprises contacting a neutrophil with an exemplary fusion polypeptide disclosed herein such that the fusion polypeptide enters the neutrophil and inhibits SNARE-associated neutrophil degranulation.

As used herein, the terms “inhibit,” “inhibition,” or grammatical variations thereof refer to any decrease or suppression of neutrophil degranulation. It is understood that the degree of inhibition need not be absolute (i.e., the degree of inhibition need not be a complete prevention of neutrophil degranulation) and that intermediate levels of inhibition of neutrophil degranulation are contemplated by the presently disclosed subject matter. As such, in some embodiments, the inhibition of neutrophil degranulation can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

Four classes of granules have been found in neutrophils, including secretory vesicles, specific granules, azurophil granules and gelatinase granules, and the determination of whether degranulation of each class of granules has been inhibited can be achieved by detecting protein markers specific for a particular granule subset on the plasma membrane of neutrophils and/or by measuring the release of the components within the granules themselves. For example, degranulation of secretory vesicles, gelatinase, granules, specific granules, and azurophil granules can be determined by measuring the plasma membrane expression of the protein markers CD35, CD11b, CD66b, and CD63, respectively, by flow cytometry. As another example, degranulation of gelatinase granules can be measured by determining the amount of gelatinase (MMP9) released using an enzyme-linked immunosorbent assay (ELISA). In some embodiments, the fusion polypeptide inhibits degranulation of a secretory vesicle, a specific granule, or a gelatinase granule.

As will be recognized by those of ordinary skill in the art, in embodiments where contacting a cell with a fusion protein of the presently-disclosed subject matter inhibits neutrophil degranulation, the optimum amount of a fusion polypeptide used to inhibit neutrophil degranulation can vary depending on the particular granule subset being inhibited as well as desired degree of inhibition. In some embodiments, neutrophil degranulation is inhibited by contacting a neutrophil with a concentration of the fusion polypeptide of about 0.5 μg/ml, about 0.8 μg/ml, about 1.0 μg/ml, about 1.5 μg/ml, about 2.0 μg/ml, about 2.5 μg/ml, about 3.0 μg/ml, about 3.5 μg/ml, about 4.0 μg/ml, about 4.5 μg/ml, about 5.0 μg/ml, about 5.5 μg/ml, about 6.0 μg/ml, about 6.5 μg/ml, about 7.0 μg/ml, about 7.5 μg/ml, about 8.0 μg/ml, about 8.5 μg/ml, about 9.0 μg/ml, about 9.5 μg/ml, or about 10.0 μg/ml. In some embodiments, a neutrophil is contacted with a concentration of the fusion polypeptide of above about 0.5 g/ml. In some embodiments, the concentration of the fusion polypeptide is about 0.8 μg/ml. Of course, determination and adjustment of the amount of a fusion polypeptide to be used in a particular application, as well as when and how to make such adjustments, can be ascertained using only routine experimentation.

As used herein, the terms “treatment” or “treating” relate to any treatment of ARDS, including but not limited to prophylactic treatment and therapeutic treatment. As such, the terms “treatment” or “treating” include, but are not limited to: preventing a neutrophil-degranulation disorder or the development of a neutrophil-degranulation disorder; inhibiting the progression of a neutrophil-degranulation disorder; arresting or preventing the development of a neutrophil-degranulation disorder; reducing the severity of a neutrophil-degranulation disorder; ameliorating or relieving symptoms associated with a neutrophil-degranulation disorder; and causing a regression of the neutrophil-degranulation disorder or one or more of the symptoms associated with the neutrophil-degranulation disorder.

As used herein, the term “subject” includes both human and animal subjects. Thus, veterinary therapeutic uses are provided in accordance with the presently disclosed subject matter. As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including racehorses), poultry, and the like.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kgx12. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species. Briefly, to express a mg/kg dose in any given species as the equivalent mg/sq m dose, multiply the dose by the appropriate km factor. In an adult human, 100 mg/kg is equivalent to 100 mg/kgx37 kg/sq m=3700 mg/m².

Suitable methods for administering to a subject a fusion polypeptide in accordance with the methods of the presently disclosed subject matter include, but are not limited to, systemic administration, parenteral administration (including intravascular, intramuscular, intraarterial administration), subcutaneous administration, and local injection. Where applicable, continuous infusion can enhance drug accumulation at a target site.

The particular mode of drug administration used in accordance with the methods of the present subject matter depends on various factors, including, but not limited to, the vector and/or drug carrier employed, the severity of the condition to be treated, and mechanisms for metabolism or removal of the drug following administration. In some embodiments of the presently disclosed methods for treating a neutrophil-mediated inflammatory disorder, an effective amount of the fusion polypeptide is administered to a subject by intravenous injection.

The term “effective amount” is used herein to refer to an amount of the therapeutic composition (e.g., a fusion polypeptide disclosed herein) sufficient to produce a measurable biological response (e.g., an inhibition of SNARE-associated degranulation in neutrophils). Actual dosage levels of active ingredients in a therapeutic composition of the presently disclosed subject matter can be varied so as to administer an amount of the fusion polypeptide(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art of medicine.

A fusion polypeptide as described herein can comprise a therapeutic composition that includes a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The therapeutic compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

DGN-23 and TAT-STX-4 exhibit several activities. DGN-23 inhibits neutrophil mobilization of secretory vesicles (SV), gelatinase granules (GG), and specific granules (SG). TAT-STX-4 inhibits SV, GG, SG, and azurophilic granule mobilization. Both TAT-SNAP-2 and TAT-STX-4 inhibit cytokine induced priming of neutrophil ROS generation in vitro and in vivo.

Both fusion proteins inhibit stimulated degranulation of secretory vesicles, gelatinase granules, and specific granules by 60% to 80%, while only TAT-STX-4 inhibits azurophilic granule degranulation by 50% to 75%. Intravenous administration of DGN-23 reduces acute lung injury in rodent models of acute respiratory distress syndrome (ARDS) and prevents proteinuria in mouse nephrotoxic nephritis. The data presented below support the use of our inhibitors of neutrophil degranulation to treat COVID-19. The attached slides summarize data on the effect of TAT-fusion proteins on COVID-19 plasma and bronchoalveolar lavage fluid (BALF) stimulation of neutrophils from healthy donors. This experimental design mimics the effect of the COVID-19 cytokine storm on circulating neutrophils in patients.

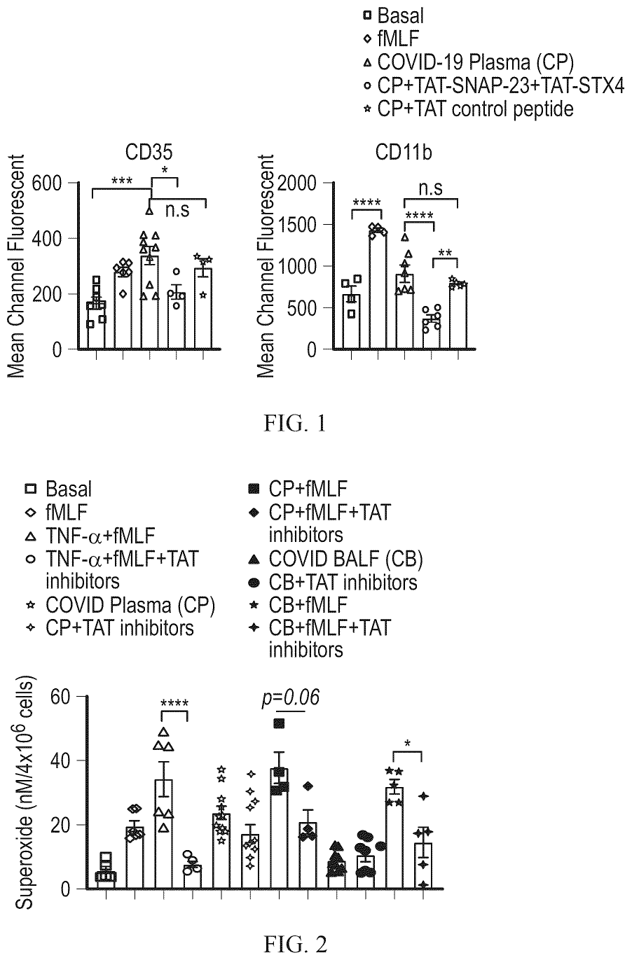

FIG. 1 confirms that plasma from patients with severe/critical COVID-19 stimulates degranulation of two neutrophil granule subsets, and that TAT-fusion proteins inhibit that stimulation. The ability of COVID-19 plasma to stimulate neutrophil degranulation is similar to that of a known pro-inflammatory stimulus, fMLF. The results show that COVID-19 plasma stimulates degranulation of secretory vesicles (CD35) and gelatinase granules (CD11b). The combination of DGN-23 and TAT-STX-4 prevented COVID-19 plasma-stimulated release of those granule subsets. A control TAT-fusion protein containing GST fused to TAT did not inhibit plasma-stimulated degranulation. These results show for the first time that COVID-19 plasma contains products that stimulate neutrophil degranulation, and our TAT-fusion protein inhibitors block that degranulation. BALF was also found to contain large quantities of cytokines and to stimulate degranulation (data not shown).

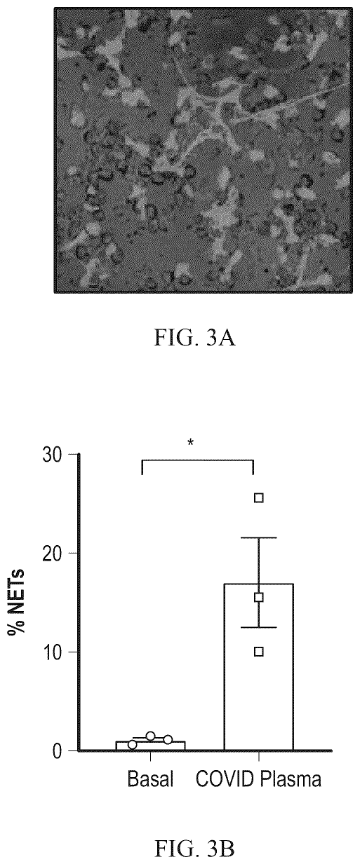

FIG. 2 summarizes experiments using different COVID-19 patient plasma and BALF to stimulate Reactive Oxygen Species (ROS) generation by neutrophils from healthy donors. These data indicate that COVID-19 plasma stimulates respiratory burst activity similar to that observed in fMLF-stimulated neutrophils, while BALF stimulates only a marginal increase in superoxide release. TAT-fusion proteins had no significant effect on ROS generation directly stimulated by plasma or BALF. Neutrophils incubated with either COVID-19 plasma or BALF were primed for enhanced fMLF-stimulated ROS generation, similar to that induced by a known priming agent, TNFα. Priming for enhanced ROS generation by TNFα, by COVID-19 plasma, and by COVID-19 BALF was significantly inhibited by TAT-fusion proteins. These data show that degranulation induced by COVID-19 plasma and BALF is a therapeutic target to prevent enhanced neutrophil ROS generation in COVID-19 patients.

Taken together, the data shown in these figures demonstrate that our compounds prevent enhanced ROS generation and release of toxic granule contents that cause acute lung injury in severely ill ARDS patients. We demonstrated that our compounds can be safely administered intravenously to rodents, and treatment of mice and rats with our compounds prevents acute lung injury induced by SARS-COV-2 infection and in 3 models unrelated to SARS-COV-2 infection.

The invention will now be illustrated by the following non-limiting Examples.

### Example 1

DGN-23, TAT-STX-4, and TAT-GST were created in 2011. They were generated by ligating the cDNA of the N-terminal SNARE domain of SNAP-23, the SNARE domain of syntaxin-4, and GST into the pTAT-vector and transformation of Escherichia coli DH5a competent cells. BL21-AI cells were transformed to overexpress the recombinant TAT fusion proteins. Purification of TAT-SNAP-23 and TAT-Control was performed by sonication and lysis of the bacterial pellet with a denaturing buffer (7 M urea, 0.5 M NaCl, 50 mM NaPO4 [pH 8], 20 mM imidazole), followed by protein separation from the supernatant by Ni-NTA beads. The ability of these fusion proteins to inhibit neutrophil degranulation was established. Subsequent reports showed that intravenous injection of DGN-23 inhibited neutrophil degranulation and respiratory burst activity in vivo and reduced acute lung injury induced by immune complex deposition, sepsis, and shock in rodents. Intravenous administration also inhibited glomerular injury induced by immune complex deposition in mice. Intravenous administration was not associated with any visible side effects in rodents. Thus, the product inhibits neutrophil function in vitro and in vivo, and this effect ameliorates inflammatory organ damage in several animal models.

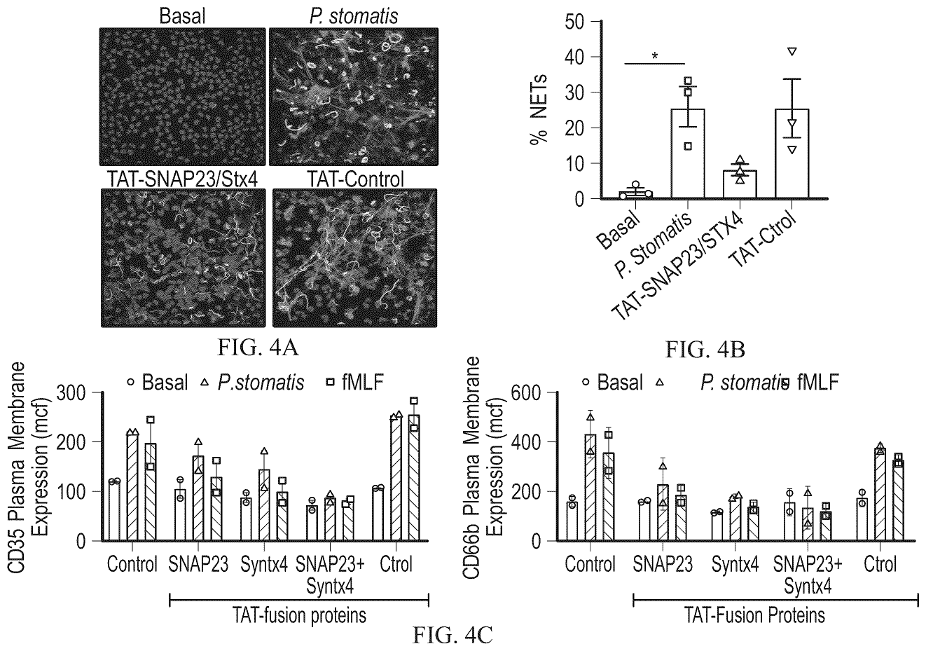

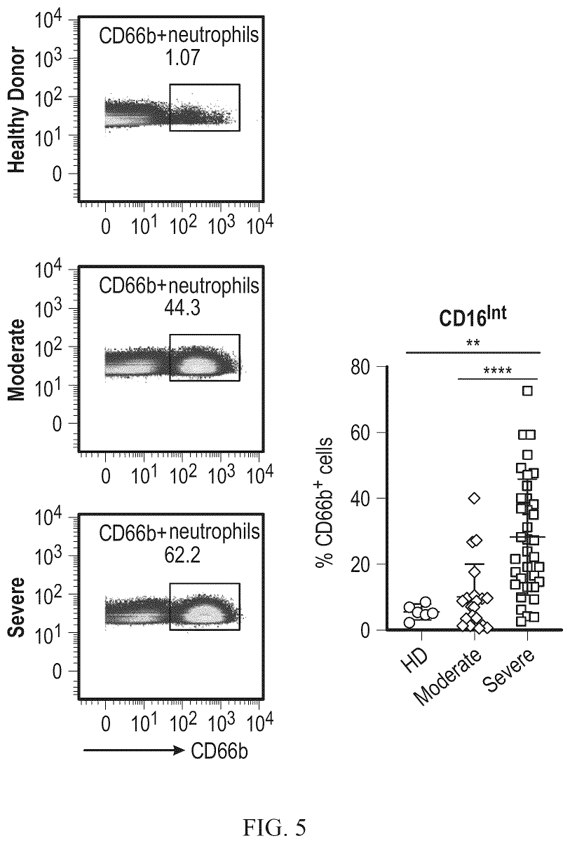

Neutrophil formation of neutrophil extracellular traps (NETs) has been shown to contribute to ARDS and coagulopathy in COVID-19 patients and participate in development of ARDS from other etiologies. FIGS. 3A-3B show that incubation of plasma from COVID-19 patients stimulates neutrophils from healthy donors to form (NETs). To evaluate the effect of TAT-fusion proteins on NET formation, normal human neutrophils were incubated with bacteria that induce NETs, P. stomatis. FIG. 4B shows that TAT-STX-4 more potently inhibits NET formation (FIG. 4B) and degranulation (FIG. 4C) than DGN-23 at the concentrations used. Thus, inhibition of degranulation has a modest inhibitory effect on NET formation.

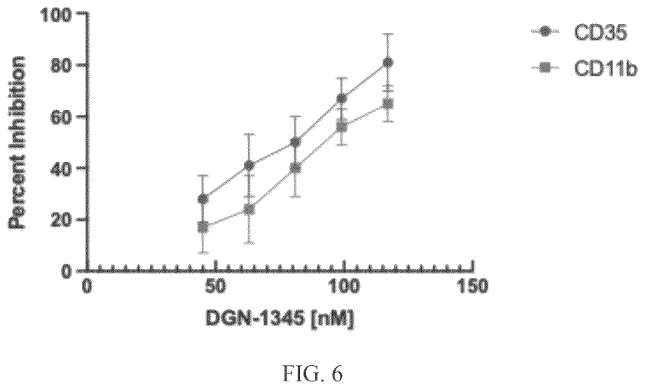

FIG. 5 shows that neutrophils undergo degranulation in vivo in COVID-19 patients, as demonstrated by the increased plasma membrane expression of a marker of specific granules, CD66b.

### Example 2

Table 1 shows the respiratory burst activity, measured as intracellular H₂O₂, stimulated by phagocytosis of killed Staphylococcus aureus for neutrophils obtained from four healthy donors and six severe COVID-19 patients.

TABLE 1
Effect of DGN-23 on Phagocytosis-Stimulated H₂O₂ Production

| | Basal (mean ± SEM) | Basal + TAT | TNF-primed | Primed + TAT |
|---|---|---|---|---|
| Healthy Donor | 396 ± 176 | 195 ± 83 | 1201 ± 237 | 372 ± 117 |
| COVID-19 Patient | 803 ± 200 | 670 ± 206 | 1940 ± 454 | 922 ± 243 |

Results presented as mean±standard error of the mean. Basal condition are neutrophils isolated from subjects without any other treatment. Basal+TAT are neutrophils pretreated for 10 minutes with DGN-23, then incubated with S. aureus. TNF-primed are neutrophils incubated with Tumor necrosis factor-α (TNFα) for 10 minutes, to prime the cells for an enhanced response, prior to addition of S. aureus. Primed+TAT neutrophils were pretreated with DGN-23 prior to priming with TNFα, then incubation with S. aureus. The results show that neutrophils from COVID-19 patients are primed in vivo for an enhanced response. DGN-23 treatment reduced the in vitro response of neutrophils from healthy donors, but it had a minimal effect on primed neutrophils from COVID-19 patients. In vitro priming with TNFα enhanced respiratory burst activity of neutrophils in both groups, and treatment with DGN-23 reduced the response back to the basal level. These data are interpreted to indicate that DGN-23 prevents the enhanced response of priming when administered prior to exposure to the priming agent. Administration of DGN-23 after priming has little or no effect on respiratory burst response. It is concluded that administration of DGN-23 to patients with COVID-19 would reduce enhanced neutrophil production of toxic oxygen radicals resulting from the “cytokine storm.” The damage to the lungs and other organs resulting from oxygen radicals are predicted to be reduced by DGN-23 treatment.

### Example 3

DGN-23 produced in BL12-AI E. coli resulted in a yield titer of less than 1 mg/liter. To obtain greater production titers, DGN-23 was expressed and purified from BL21DE3 or BL21DE3 pLysS. Although the titer achieved was up to 10 mg/liter, purified DGN-23 did not always inhibit neutrophil degranulation. Evaluation of DGN-23 purity by SDS-PAGE and immunoblot analysis identified a 15 kDa protein expressed in inactive batches, in addition to the predicted 25 kDa protein. Immunoblotting determined that both the 15 kDa and 25 kDa bands contained a His tag at the C-terminus, a HA tag within the central portion of the protein, and the SNARE domain from the N-terminus. It was concluded that the 15 kDa protein resulted from partial proteolysis of the N-terminal SNARE domain, leading to inactivation of the protein. Inclusion of HALT proteases in the cell lysis buffer modestly reduced the 15 kDa protein generation, but did not result in recovery of inhibitory activity, suggesting that degradation and inactivation were occurring during the protein induction phase. Subsequently, a time course of bacterial growth and protein induction in the presence of IPTG and arabinose was performed during expression of DGN-23 in BL21-AI cells. By decreasing the induction time and optimizing growth rate, expression of the 15 kDa protein band was reduced, but not eliminated. Additionally, titers obtained were less than 1 mg/liter.

To produce DGN-23 at higher titers that also effectively inhibited neutrophil degranulation, recombinant proteins containing shorter fragments of the SNAP-23 SNARE domain were generated. Of the 78 amino acids in that SNARE domain, modified DGN-23 containing amino acids 13-45, 13-78, 19-78 or 46-78 were produced. These modified DGN-23 proteins were expressed in BL21-AI cells, and purified by Ni bead chromatography. Protein separation by SDS-PAGE and immunoblot analysis for HA and SNAP-23 of the recombinant protein containing amino acids 13-45 were performed. Importantly, this protein was at the appropriate molecular size, contained the HA tag and SNAP-23 SNARE domain, and no degradation products were present.

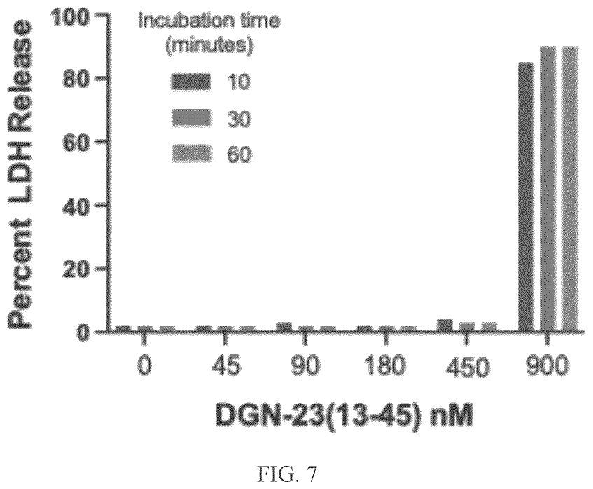

FIG. 6 shows that modified DGN-23, containing amino acids 13-45 of the SNARE domain (termed DGN-1345), inhibited neutrophil degranulation with concentration inhibition curves similar to the parent compound. DGN-23 containing amino acids 19-78 and 46-78 failed to inhibit neutrophil degranulation. Yield of DGN-1345 obtained without further optimization of production ranged from a titer of 12 to 28 mg/liter. The yield and reliable in vitro potency of DGN-1345 led to further modifications of this lead product. Those additional modifications consisted of elimination of the His tag, due to potential in vivo toxicity if metal binding, and of the HA tag, due to potential enhanced immunogenicity. The final amino acid composition of the lead DGN-1345 product is the following:

(SEQ ID NO: 15)
YGRKKRRQRRRAHQITDESLESTRRILGLAIESQDAGIKTITML

The TAT permeability peptide sequence is in bold (SEQ ID NO: 5) and the 13-45 amino acid sequence of the SNAP-23 N-terminal SNARE domain is italicized (SEQ ID NO: 16).

In addition to assessing purity of DGN-1345 by SDS-PAGE and immunoblot analysis for SNAP-23 and HA tag, two batches were subjected to analysis by HPLC and subjecting peaks identification by mass spectrometry. The major peak accounted for 95%-96% of the total material present. Peptides covering the entire 13-45 SNAP-23 sequence were identified by MS. The total peptide coverage of the molecule was 66%, with the TAT permeability sequence and His tag regions accounting for unidentified portions of the molecule. No contaminant accounted for more than 1% of total protein, and MS determined that all contaminating proteins were derived from E. coli.

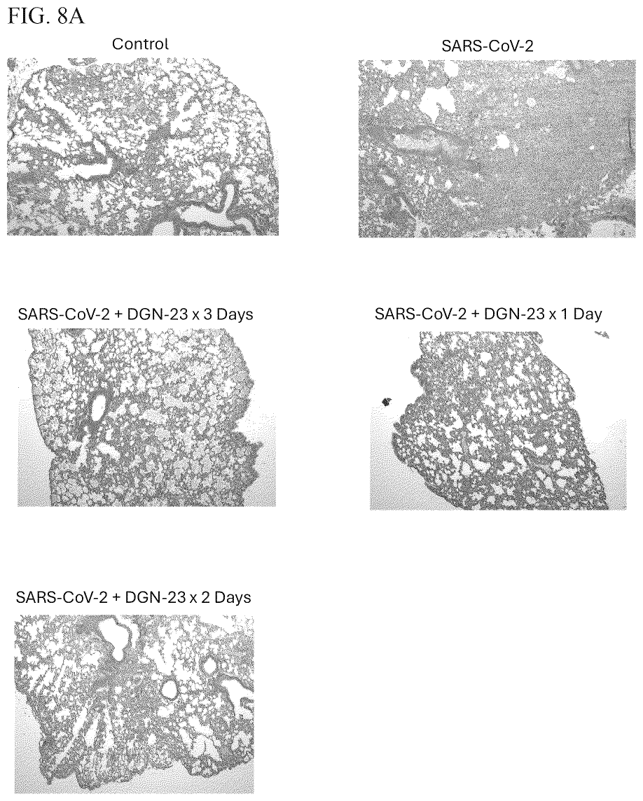

In vitro cytotoxicity of DGN-23 and DGN-1345 was performed by measuring LDH release into supernatants from neutrophil degranulation assays and from cultured HK-11 cells incubated with various concentrations of DGN-1345. Human neutrophils incubated for 45 min with or without 45 nM to 140 nM DGN-1345, in the presence or absence of stimulation with fMLF, showed release of 1% to 6% of total LDH with no dose effect. FIG. 7 shows the results of incubation of cultured HK-11 cells with DGN-1345 for 10 to 60 min at concentrations of 0 nM to 900 nM. Increased LDH was only observed at the highest concentration tested. Those data indicate that DGN-1345 demonstrates in vitro cell cytotoxicity only at concentrations >10 times higher than those that inhibit neutrophil degranulation. Importantly, DGN-1345 shows no evidence of neutrophil toxicity at concentrations that inhibit degranulation.

The ability of human neutrophils to ingest and kill the oral pathogen F. alocis, in vitro, in the presence or absence of DGN-C or concentrations of DGN-23 and DGN-1345 that inhibit degranulation was determined u. Bacterial survival in the presence of untreated neutrophils was 21+/−5% (n=4), with DGN-C 38+/−24%, with DGN-23 26+/−7%, and with DGN-1345 23+/−7%. Thus, inhibition of neutrophil degranulation with DGN-23 or DGN-1345 does not inhibit in vitro phagocytosis and killing of bacteria.

The effect of DGN-23 administration initiated 1, 2, or 3 days after SARS-COV-2 infection on lung injury is shown in FIGS. 8A-8G. FIG. 8A shows representative photomicrographs of lung histology from the 5 groups of mice. Statistical analysis of the lung injury score is shown in FIG. 8B. While the injury score was lower in all DGN-23 treatment groups, differences were statistically significant only when DGN-23 administration was started on day one after infection. As this injury score is partially determined by neutrophil lung infiltration, which is not impaired by DGN-23, the histology score may not adequately reflect the value of DGN-23 treatment. Therefore, alveolar-capillary permeability was assessed by protein content of bronchoalveolar lavage fluid (BALf) performed at the time of euthanasia and by lung weight. FIGS. 8C and 8D show that DGN-23 administration significantly inhibited increased alveolar-capillary permeability associated with lung injury even when started 3 days after infection. DGN-23 treatment prevented injury to lung epithelial cells, as shown by the inhibition of RAGE release into BALf (FIG. 8E). Importantly, ELISA of BALf for MMP-9 (from gelatinase granules) and elastase (from azurophilic granules) showed that DGN-23 inhibited degranulation by recruited neutrophils up to 24 hr after the last dose (FIGS. 8F and 8G).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A fusion protein comprising a TAT-fusion protein comprising an 11 amino acid cell penetrating peptide TAT operably linked to an amino acid N-terminal SNARE domain of SNAP-23, wherein the N-terminal SNARE domain of SNAP-23 is about 30 to 70 amino acids in length.

2. The fusion protein of claim 1, wherein the cell penetrating peptide TAT is amino acid sequence YGRKKRRQRRR (SEQ ID NO: 5).

3. The fusion protein of claim 1 or 2, wherein the N-terminal SNARE domain of SNAP-23 is amino acid sequence AHQITDESLESTRRILGLAIESQDAGIKTITML (SEQ ID NO: 16), MAHQITDESLESTRRILGLAIESQDAGIKTITML (SEQ ID NO: 17) or
MAHQITDESLESTRRILGLAIESQDAGIKTITMLDEQKEQLNRIEEGLDQINKD MRETEKTLTELNK (SEQ ID NO:18).

4. The fusion protein of any one of claims 1-3, wherein the protein is amino acid sequence YGRKKRRQRRRAHQITDESLESTRRILGLAIESQDAGIKTITML (SEQ ID NO: 15).

5. A pharmaceutical composition comprising the fusion protein of any one of claims 1-4 and a pharmaceutically acceptable excipient.

6. A method inhibiting neutrophil degranulation comprising administering the fusion protein of any one of claims 1-4 or the pharmaceutical composition of claim 5, to a patient in need thereof, wherein the inhibition is compared to a control.

7. A method inhibiting generation of reactive oxygen species (ROS) by neutrophils comprising administering the fusion protein of claim 1 to a patient in need thereof, wherein the inhibition is compared to a control.

8. A method inhibiting release of intracellular granule contents (degranulation) by neutrophils comprising administering the fusion protein of claim 1 to a patient in need thereof, wherein the inhibition is compared to a control.

9. The method of any one of claims 6-8, wherein the patient has acute respiratory distress syndrome (ARDS) or the patient is infected with severe acute respiratory syndrome coronavirus 2 (SARS-COV-2).


SEQUENCE LISTING (23 sequences)

SEQ ID NO: 1 - protein, 78 aa, Homo sapiens
MDNLSSEEIQQRAHQITDESLESTRRILGLAIESQDAGIKTITMLDEQKEQLNRIEEGLDQINKDMRETEKTLTELNK

SEQ ID NO: 2 - protein, 40 aa, Homo sapiens
TITMLDEQKEQLNRIEEGLDQINKDMRETEKTLTELNKCC

SEQ ID NO: 3 - protein, 60 aa, Homo sapiens [sequence omitted]

SEQ ID NO: 4 - protein, 24 aa, Homo sapiens
TDKADTNRDRIDIANARAKKLIDS

SEQ ID NO: 5 - protein, 11 aa, unidentified
  source 1..11: note = Human immunodeficiency virus
YGRKKRRQRRR

SEQ ID NO: 6 - protein, 16 aa, synthetic construct
AKIWFQNRRMKWKKEN

SEQ ID NO: 7 - skipped

SEQ ID NO: 8 - protein, 9 aa, synthetic construct
RRRRRRRRR

SEQ ID NO: 9 - protein, 21 aa, synthetic construct
KETWWETWWTEWSQPKKKRKV

SEQ ID NO: 10 - protein, 27 aa, synthetic construct
GWTLNSAGYLLGKINLKALAALAKKIL

SEQ ID NO: 11 - DNA, 438 bp, synthetic construct
  source 1..438: mol_type = other DNA
atgcggggttctcatcatcatcatcatcatggtatggctagcatgactggtggacagcaaatgggtcgggatctgtacgacgatgacgataaggatcgatggggatccaagcttggctacggccgcaagaaacgccgccagcgccgccgcggtggatccaccatgtccggctatccatatgacgtcccagactatgctggctccatggataatctgtcatcagaagaaattcaacagagagctcaccagattactgatgagtctctggaaagtacgaggagaatcctgggtttagccattgagtctcaggatgcaggaatcaagaccatcactatgctggatgaacaaaaggaacaactaaaccgcatagaagaaggcttggaccaaataaataaggacatgagagagacagagaagactttaacagaactcaacaaa

SEQ ID NO: 12 - protein, 146 aa, synthetic construct
MRGSHHHHHHGMASMTGGQQMGRDLYDDDDKDRWGSKLGYGRKKRRQRRRGGSTMSGYPYDVPDYAGSMDNLSSEEIQQRAHQITDESLESTRRILGLAIESQDAGIKTITMLDEQKEQLNRIEEGLDQINKDMRETEKTLTELNK

SEQ ID NO: 13 - protein, 9 aa, synthetic construct
YPYDVPDYA

SEQ ID NO: 14 - protein, 68 aa, synthetic construct
VTRQALNEISARHSEIQQLERSIRELHDIFTFLATEVEMQGEMINRIEKNILSSADYVERGQEHVKTA

SEQ ID NO: 15 - protein, 44 aa, synthetic construct
YGRKKRRQRRRAHQITDESLESTRRILGLAIESQDAGIKTITML

SEQ ID NO: 16 - protein, 33 aa, Homo sapiens
AHQITDESLESTRRILGLAIESQDAGIKTITML

SEQ ID NO: 17 - protein, 34 aa, synthetic construct
MAHQITDESLESTRRILGLAIESQDAGIKTITML

SEQ ID NO: 18 - protein, 67 aa, synthetic construct
MAHQITDESLESTRRILGLAIESQDAGIKTITMLDEQKEQLNRIEEGLDQINKDMRETEKTLTELNK

SEQ ID NO: 19 - protein, 33 aa, Homo sapiens
DEQKEQLNRIEEGLDQINKDMRETEKTLTELNK

SEQ ID NO: 20 - DNA, 132 bp, synthetic construct
  source 1..132: mol_type = other DNA
tacggccgcaagaaacgccgccagcgccgccgcgctcaccagattactgatgagtctctggaaagtacgaggagaatcctgggtttagccattgagtctcaggatgcaggaatcaagaccatcactatgctg

SEQ ID NO: 21 - protein, 34 aa, Homo sapiens
RAHQITDESLESTRRILGLAIESQDAGIKTITML

SEQ ID NO: 22 - protein, 66 aa, Homo sapiens
AHQITDESLESTRRILGLAIESQDAGIKTITMLDEQKEQLNRIEEGLDQINKDMRETEKTLTELNK

SEQ ID NO: 23 - protein, 60 aa, Homo sapiens
ESLESTRRILGLAIESQDAGIKTITMLDEQKEQLNRIEEGLDQINKDMRETEKTLTELNK